(19) United States
(12) Patent Application Publication
(10) Pub. No.: US 2023/0193225 A1
(43) Pub. Date: Jun. 22, 2023

(54) NEW USES OF A MUTATED LACTONASE, AND COMPOSITIONS

(71) Applicants: FONDATION MEDITERRANEE INFECTION, MARSEILLE (FR); GENE AND GREEN TK, MARSEILLE (FR); AIX-MARSEILLE UNIVERSITE, MARSEILLE CEDEX 07 (FR)

(72) Inventors: Eric CHABRIERE, AUBAGNE (FR); Sonia MION, AIX-EN-PROVENCE (FR); Benjamin REMY, MARSEILLE (FR); Laure Claude PLENER, MARSEILLE (FR); David DAUDE, MARSEILLE (FR); Mikael Hocine ELIAS, MINNEAPOLIS, MN (US)

(21) Appl. No.: 17/440,670

(22) PCT Filed: Mar. 16, 2020
(86) PCT No.: PCT/EP2020/057159

§ 371 Date: Sep. 17, 2021

(30) Foreign Application Priority Data
Mar. 19, 2019 (FR) FR1902834

(51) Int. Cl. C12N 9/16; A01N 63/50; A61P 31/04; A61L 2/00

(52) U.S. Cl. CPC C12N 9/16; C12Y 301/08; A01N 63/50; A61P 31/04; A61L 2/0088

(57) ABSTRACT

Disclosed is a mutated lactonase belonging to the phosphotriesterase-like lactonase family, which increases the susceptibility of bacteria to antimicrobial agents as compared to the use of antimicrobial agents alone.

The present invention relates to new uses of a mutated lactonase and compositions containing it.

Some bacteria use a molecular communication system called Quorum Sensing (QS) to coordinate many biological functions such as virulence or biofilm formation. In particular, they use acyl-homoserine lactones (AHL) as communication molecules.

The formation of bacterial biofilm causes both medical and environmental problems. It is therefore important to find solutions to effectively remove bacterial biofilms.

Thus, in a first aspect, the invention relates to the use of a mutated lactonase to increase the susceptibility of bacteria to antimicrobial agents.

In a second aspect, the invention relates to the use of a mutated lactonase and at least one antimicrobial agent to inhibit bacterial growth.

In a third aspect, the invention concerns the use of a mutated lactonase to increase the sensitivity of bacteria to bacteriophages.

In a fourth aspect, the invention relates to compositions comprising a mutated lactonase.

In a fifth aspect, the invention relates to a method for the prevention and/or treatment of pathologies related to bacterial infections.

Thus, the invention relates in particular to the use of a mutated lactonase belonging to the phosphotriesterase-like lactonase family to increase the susceptibility of bacteria to antimicrobial agents compared to the use of antimicrobial agents alone, in which at least the amino acid tryptophan at the beginning of loop 8 is substituted by the amino acid isoleucine, and in particular, said mutated lactonase having the sequence SEQ ID NO: 1 in which at least the amino acid tryptophan W at position 263 is substituted by the amino acid isoleucine I, and in particular, said sensitivity of bacteria to antimicrobial agents is increased by at least a factor of 2.

Surprisingly, the Inventors of the present application have found that the use of a mutated hyperthermophilic lactonase increases the sensitivity of bacteria to antimicrobial agents, thus allowing effective targeting of the biofilm.

In all aspects of the present invention, said mutated lactonase is derived from the hyperthermophilic lactonase of Sulfolobus solfataricus (SsoPox) or Saccharolobus solfataricus. Hyperthermophilic lactonase from Sulfolobus solfataricus (SsoPox) or Saccharolobus solfataricus has both phosphotriesterase and lactonase activity. It belongs to the phosphotriesterase-like lactonase family

Enzymes of the phosphotriesterase-like lactonase family have a conserved three-dimensional structure. Thus, according to the present invention, said mutated lactonase comprises at least the substitution of the amino acid tryptophan located at the beginning of loop 8 of the enzymes of the Phosphotriesterase-Like Lactonase family by the amino acid Isoleucine.

According to the present invention, the mutated lactonase may comprise other mutations, in addition to the tryptophan mutation located at the beginning of loop 8 of the Phosphotriesterase-Like Lactonase family of enzymes. These additional mutations may, for example, improve the properties of the mutated lactonase or improve its stability.

Thus, the present invention relates in particular to the use of a mutated lactonase belonging to the phosphotriesterase-like lactonase family to increase the susceptibility of bacteria to antimicrobial agents compared to the use of antimicrobial agents alone,

in which at least the amino acid tryptophan at the beginning of loop 8 is substituted by the amino acid isoleucine.

According to a particularly preferred embodiment, the mutated lactonase of the invention is derived from the hyperthermophilic lactonase of Sulfolobus solfataricus (SsoPox) having the sequence SEQ ID NO: 1 in which the amino acid tryptophan at position 263 is substituted by an Isoleucine I. Thus, in this embodiment, the mutated lactonase has the sequence SEQ ID NO: 2 and has a mutation at position 263 relative to the sequence SEQ ID NO: 1.

| | |
|---|---|
| SsoPox | MRIPLVGKDSIESKDIGFTLIHEHLRVFSEAVRQQWPHL |
| Wild- | YNEDEEFRNAVNEVKRAMQFGVKTIVDPTVMGLGRDIRF |
| type | MEKVVKATGINLVAGTGIYIYIDLPFYFLNRSIDEIADL |
| SEQ ID | FIHDIKEGIQGTLNKAGFVKIAADEPGITKDVEKVIRAA |
| NO: 1 | AIANKETKVPIITHSNAHNNTGLEQRILTEEGVDPGKIL |
| | IGHLGDTDNIDYIKKIADKGSFIGRYGLDLFLPVDKRNE |
| | TTLRLIKDDGYSDKIMISHDYCCTIDWGTAKPEYKKPLA |
| | PRWSITLIFEDTIPFLKRNGNEEVIATIFKENPKKFFS |
| W263I | MRIPLVGKDSIESKDIGFTLIHEHLRVFSEAVRQQWPHL |
| mutated | YNEDEEFRNAVNEVKRAMQFGVKTIVDPTVMGLGRDIRF |
| SsoPox | MEKVVKATGINLVAGTGIYIYIDLPFYFLNRSIDEIADL |
| SEQ ID | FIHDIKEGIQGTLNKAGFVKIAADEPGITKDVEKVIRAA |
| NO: 2 | AIANKETKVPIITHSNAHNNTGLEQRILTEEGVDPGKIL |
| | IGHLGDTDNIDYIKKIADKGSFIGRYGLDLFLPVDKRNE |
| | TTLRLIKDDGYSDKIMISHDYCCTIDIGTAKPEYKPKLA |
| | PRWSITLIFEDTIPFLKRNGNEEVIATIFKENPKKFFS |

Thus, in this particular embodiment, the present invention relates to the use of a mutated lactonase to increase the susceptibility of bacteria to antimicrobial agents as compared to the use of antimicrobial agents alone,

said mutated lactonase having the sequence SEQ ID NO: 1 in which amino acid W at position 263 is substituted by amino acid isoleucine I.

Thus, in this case, the tryptophan at the beginning of loop 8 corresponds to the residue at position 263 of the primary structure. However, in the case of other phosphotriesterase-like lactonase enzymes, it may be a different position in the primary structure, but it will always be the tryptophan residue at the beginning of loop 8 of the Phosphotriesterase-Like Lactonase enzymes.

According to the present invention, and in this embodiment, the mutated lactonase comprises at least the substitution of tryptophan by isoleucine at position 263 of its sequence, as shown in SEQ ID NO: 2. Indeed, according to the present invention, the mutated lactonase may comprise other mutations, in addition to the mutation at position 263 of its sequence. These additional mutations may, for example, improve the properties of the mutated lactonase of sequence SEQ ID NO: 2 or improve its stability.

Thus, the present invention also relates to the use of a mutated lactonase to increase the susceptibility of bacteria to antimicrobial agents compared to the use of antimicrobial agents alone,

said mutated lactonase having the sequence SEQ ID NO: 1 in which at least amino acid W at position 263 is substituted by amino acid isoleucine I.

Thus, according to the present invention, when a given anti-microbial agent is used in combination with the mutated lactonase as defined above and in particular with the mutated lactonase of sequence SEQ ID NO: 2, the sensitivity of the bacteria to this anti-microbial agent is increased compared to the use of the same anti-microbial agent alone. This means that the amount of anti-microbial agent required to eliminate or inhibit the growth of these bacteria can be reduced when the anti-microbial agent is used with the mutated lactonase as defined above and in particular with the mutated lactonase of sequence SEQ ID NO: 2. Thus, the toxicity associated with the use of potentially high concentrations of anti-microbial agent is reduced. This also avoids the development of resistance mechanisms to antimicrobial agents by bacteria.

According to the present invention, in order to measure the susceptibility of bacteria to antimicrobial agents, it is possible to measure, according to methods known to the skilled person:
the Minimum Inhibition Concentration (MIC or CMI in french)
the Minimum Biofilm Eradication Concentration (MBEC or CMEB in french)
the relative abundance of proteins involved in resistance to antimicrobial agents (efflux pumps, porins)
the expression of genes in the CRISPR-Cas system involved in bacteriophage resistance.

Thus, for a given dose of antimicrobial agent, the observation of a decrease in the minimum inhibition concentration, a decrease in the minimum biofilm eradication concentration, a variation in the relative abundance of proteins involved in resistance to antimicrobial agents or an alteration in the expression of genes of the CRISPR-Cas system involved in bacteriophage resistance means that the sensitivity of bacteria to antimicrobial agents is increased.

In an embodiment of this first aspect, the susceptibility of bacteria to the antimicrobial agents is increased by at least a factor of 2 compared to the use of said antimicrobial agents alone.

This means that, according to the present invention, the sensitivity of bacteria to antimicrobial agents is increased by a factor of 2, 3, 4, 5, 10, 20, 30, 40, 50, 60, 70, 80, 90 or 100 compared to the use of said antimicrobial agents alone.

In a particularly preferred embodiment, the susceptibility of bacteria to the anti-microbial agents is increased by a factor of between 2 and 20 compared to the use of said anti-microbial agents alone.

In all aspects of the present invention, the term “antimicrobial agent” refers to a compound that kills a microorganism or inhibits its growth.

In a particular embodiment, an antimicrobial agent may, for example, be an antibacterial agent, an antifungal agent, an antiviral agent, an antiprotozoal agent, an antiparasitic agent or a combination thereof.

An antimicrobial agent within the meaning of the present invention may, for example, be an inorganic compound, an organic compound, a protein, an antibody, a sugar, a nucleic acid or a combination thereof.

In a particular embodiment, the anti-microbial agent may be selected from the group consisting of antibiotics or a mixture of antibiotics, disinfectants or a mixture of disinfectants, biocides or a mixture of biocides, and bacteriophages, which may or may not be naturally present in the environment, or a cocktail of such bacteriophages.

Thus, the invention relates in particular to the use as described above, wherein the antimicrobial agent is selected from the group consisting of antibiotics or a mixture of antibiotics, disinfecting agents or a mixture of disinfecting agents, biocides or a mixture of biocides and bacteriophages possibly naturally present in the environment or not, or a cocktail of such bacteriophages.

In all aspects of the invention, “antibiotic” means any agent capable of killing a bacterium or reducing, limiting or inhibiting its growth.

According to the present invention, the antibiotics may be bactericidal antibiotics or bacteriostatic antibiotics. “Bactericidal antibiotics” means any agent capable of killing a bacterium. “Bacteriostatic antibiotics” means any agent capable of reducing, limiting or inhibiting bacterial growth, without killing the bacteria.

In all aspects of the invention, a “disinfectant” is any substance applied to a non-living (inert) or living object (such as the skin) and capable of killing or inhibiting the growth of microorganisms present on the object. A disinfectant for body use, i.e. applied to the external surfaces of the body, such as the skin, is called an “antiseptic”.

In all aspects of the invention, “biocide” means any substance or preparation intended to destroy, repel or render harmless harmful organisms, to prevent the action of harmful organisms or to combat them, by chemical or biological action. In other words, biocides are substances that act on or against harmful organisms.

In all aspects of the invention, “bacteriophage” means any virus capable of infecting bacteria. Two types of bacteriophages can be distinguished:
lytic phages that infect the bacteria, hijack its cellular machinery to reproduce and destroy the cell to release new phages
Lysogenic, or temperate phages, which insert their DNA into the bacterium in the form of a prophage.

In all aspects of the present invention, “bacteriophages which may or may not be naturally present in the environment” means bacteriophages naturally present in the environment as well as bacteriophages not present in the environment which are added by a third party in order to eliminate bacteria.

In a particular embodiment, said antibiotic may be selected from the group consisting of: Amikacin, Amoxicillin, Amoxicillin/clavulanate, Ampicillin, Amprolium, Apramycin, Aspoxicillin, Aureomycin, Avilamycin, Azithromycin, Bacitracin, Bambermycin, Baquiloprim, Benzylpenicillin, Bicozamycin, Carbadox, Cefacetrile, Cefalexin, Cefalonium, Cefalotin, Cefapyrin, Cefazolin, Cefdinir, Cefquinome, Ceftiofur, Ceftriaxone, Cefuroxime, Chloramphenicol, Chlortetracycline, Ciprofloxacin, Clarithromycin, Clindamycin, Cloxacillin, Colistin, Dalbavancin, Danofloxacin, Decoquinate, Diclazuril, Dicloxacillin, Difloxacin, Doripenem, Doxycycline, Enramycin, Enrofloxacin, Ertapenem, Erythromycin, Florfenicol, Flumequine, Fosfomycin, Framycetin, Fusidic acid, Gentamicin, Gentamicin Sulfate, Gramicidin, Halofuginone hydrobromide, Hetacillin, Imipenem, Imipenem/cilastatin, Josamycin, Kanamycin, Kitasamycin, Laidlomycin, Lasalocid , Levofloxacin, Lincomycin, Lincomycin hydrochloride, Maduramycin, Marbofloxacin, Mecillinam, Meropeneme, Miloxacin, Minocycline, Mirosamycin, Monensin, Moxifloxacin, Nafcillin, Nalidixic acid, Narasin, Neomycin, Neomycin/oxytetracycline, Neosporin, Nicarbazine, Norfloxacin, Novobiocin, Ofloxacin, Orbifloxacin, Oritavancin, Oxacillin, oxolinic acid, Oxytetracycline, Paromomycin, penethamate hydroxide, Penicillin, Penicillin G Potassium, Penicillin procaine, Penicillin V potassium, Phenethicillin, Phenoxymethylpenicillin, Pirlimycin, Polymyxin, Polymyxin B, Polysporin (bacitracin/polymyxin), Pristinamycin, Rifampin, Rifaximin, Roxars one, Salinomycin, Semduramicin, Spectinomycin, Spiramycin, Streptomycin, Sulfachlorpyridazine, Sulfadiazine, Sulfadimerazine, Sulfadimethoxazole, Sulfadimethoxine, Sulfadimethoxine and ormetoprim 5:3, Sulfadimidine, Sulfadoxine, Sulfafurazole, Sulfaguanidine, Sulfamethazine, Sulfamethoxazole/trimethoprim, Sulfamethoxine, Sulfamethoxypyridazine, Sulfamonomethoxine, Sulfanilamide, Sulfaquinoxaline, Sulfas alazine, Sulfisoxazole, Surfactin, Telavancin, Terdecamycin, Tetracycline, Thiamphenicol, Tiamulin, Ticarcillin, Tilmicosin, Tobicillin, Tobramycin, Trimethoprim, Trimethoprim/Sulfonamide, Tulathromycin, Tylosin, V alnemulin, Vancomycin, Virginiamycin.

In a particular embodiment, said disinfecting agent may comprise an alcohol, chlorine, aldehyde, oxidising agent, iodine, ozone, phenolic compound, quaternary ammonium compound or a mixture of two or more thereof.

In a particular embodiment, said disinfecting agent may comprise formaldehyde, orthophthalaldehyde, glutaraldehyde, silver dihydrogen citrate, polyaminopropyl biguanide, sodium bicarbonate, lactic acid, chlorine bleach, methanol, ethanol, n-propanol, 1-propanol, 2-propanol, isopropanol, hypochlorite, chlorine dioxide, dichloro isocyanurate, mono chloro isocyanurate, hydantoin, sodium hypochlorite, calcium hypochlorite, sodium dichloro isocyanurate, sodium chlorite, 4-methylbenzenesulfonamide, sodium salt, 2,4-dichlorobenzyl alcohol, performic acid, paracetic acid, potassium permanganate, potassium peroxymonosulphate, phenol, phenylphenol, chloroxylenol, hexachlorophene, thymol, amylmetacresol, benzalkonuim chloride, cetyltrimethylammonium bromide, cetylpyridinium chloride, benzethonium chloride, boric acid, brilliant green, chlorhexidine gluconate, iodine providone, mercurochrome, manuka honey, octenidine dihydrochloride, polyhexamethylene biguanide, balsam of Peru, hydrogen peroxide, organic peroxide, peroxyacid, organic hydroperoxide, salt of peroxide, acid peroxides and mixtures of two or more of these.

In a particular embodiment, said biocide may be selected from the group consisting of: biocidally active peroxides such as hydrogen peroxide, mono- and polyhydric alcohols, aldehydes, acids, ozone, naphtha compounds, and compounds containing an alkali metal, transition metal, Group III or Group IV metal, sulphur, nitrogen or halogen atom, and mixtures of two or more of these.

In a particular embodiment, said biocide is selected from the group consisting of: formaldehyde, glutaraldehyde, peracetic acid, alkali metal hypochlorites, quaternary ammonium compounds, 2-amino-2-methyl-1-propanol, cetyltrimethylammonium bromide, cetylpyridinium chloride, 2,4,4-trichloro-2-hydroxy diphenyl ether, 1-(4-chlorophenyl)-3-(3,4-dichlorophenyl) urea, zinc oxide, zinc ricinoleate, pentachlorophenol, copper naphthenate, tributyltin oxide, dichlorophene, p-nitrophenol, p-chloro-m-xylenol, beta-naphthol, 2,3,5,6-tetrachloro-4-(methylsulfonyl) pyridine, salicylanilide, bromoacetic acid, alkyl quaternary ammonium acetate, sodium ethyl mercury thiosalicylate, sodium orthophenylphenate, n-alkyl (C2 to Cs) dimethyl benzyl ammonium chloride, organoborates, 2,2-(1-methyltrimethylene dioxy)-bis-(4-methyl-1,3,2-dioxaborinane), 2,2-oxybis (4,4, 6-trimethyl)-1,3,2-dioxaborinane, ethylene glycol monomethyl ether, parahydroxybenzoates, organic boron compounds, 8-hydroxyquinoline, sodium pentachlorophenate, alkyl dimethyl ethyl benzyl ammonium chloride, alkylammonium salts, 1,3,5-triethylhexahydro-1,3,5-triazine, strontium chromate, halogenated phenols, 2-bromo-4-phenyl phenol, silver salts such as silver nitrate, silver chloride, silver oxide and elemental silver, organic peroxides, silver sulphadiazine, sodium dichloro-S-triazinetrione, 4-chloro-2-cyclohexylphenol, 2-chloro-4-nitrophenol, paraffin substitutes, 3-chloro-3-nitro-2-butanol, 2-chloro-2-nitro-1-butanol stearate, 2-chloro-2-nitrobutyl acetate, 4-chloro-4-nitro-3-hexanol, 1-chloro-1-nitro-1-propanol, 2-chloro-2-nitro-1-propanol, triethyltin chloride, 2,4,5-trichlorophenol, 2,4,6-trichlorophenol, 1,3-dichloro-5,5-dimethylhydantoin, tris(hydoxymethyl)nitromethane, nitroparaffins, 2-nitro-2-ethyl-1,3-prop anediol, 2-ethyl-2-nitro-1,3-propanediol, 2-methyl-2-nitro-1,3-propanediol, hexahydro-1,3,5-tris (2-hydroxyethyl)-S-triazine, hexahydro-1,3,5-tris(tetrahydro-2-furanyl)-methyl-S-triazine, methylene bis(thiocyanate), 2,2-dibromo-3-nitrilopropionamide, Beta-bromo-3-nitrostyrene, fluorine compounds, N-ethyl-N-methyl-4-(trifluoromethyl)-2-(3,4-dimethoxyphenyl) benzamide, pentachlorophenol, dichlorophene, orthophenylphenol, di-bicyclo (3,1,1 or 2,2,1)-heptyl polyamines, di-bicyclo-(3,1,1 or 2,2,1)-heptanyl polyamines and mixtures of two or more of these.

In a particular embodiment, said bacteriophage may belong to the family Myoviridae, Siphoviridae, Podoviridae, Corticoviridae, Cystoviridae, Inoviridae, Leviviridae, Microviridae, Plasmaviridae and Tectiviridae or a cocktail thereof.

According to the present invention, the nature of the antimicrobial agent to be used depends on the nature of the bacteria to be eliminated.

In the present invention, the term “bacteria” refers to a genus of prokaryotic microorganisms scientifically classified as such. Most bacteria can be classified as gram-positive or gram-negative.

Thus, in one particular embodiment, the bacteria may be selected from gram-positive bacteria and gram-negative bacteria.

According to the present invention, “Gram-positive bacteria” are bacteria bound by a single lipid membrane and containing a thick layer of peptidoglycans (20-80 nm) that retain crystal violet staining in a Gram staining technique.

According to the present invention, “Gram-negative bacteria” are bacteria bound by a cytoplasmic membrane as well as by an outer cell membrane, containing only a thin layer of peptidoglycans between the two membranes, which does not allow the retention of crystal violet dye in a Gram staining technique.

More particularly, said bacteria may be selected from the group consisting of: Acinetobacter baumannii, Aerococcus viridans, Aeromonas caviae, Aeromonas hydrophila, Aeromonas jandaei, Aeromonas salmonicida, Aeromonas sobria, Aeromonas veronii, Agrobacterium tumefaciens, Aliivibrio fischeri, Aliivibrio salmonicida, Brucella abortus, Brucella canis, Brucella melitensis, Brucella suis, Burkholderia cepacia complex, Burkholderia pseudomallei, Burhkolderia mallei, Chlamydia trachomatis, Chromobacterium violaceum, Clavibacter michiganensis, Clostridium botulinum, Clostridium difficile, Comamonas acidovorans, Comamonas testosteronii, Delftia acidovorans, Desulfovibrio desulfuricans, Desulfovibrio gigas, Desulfovibrio vulgaris, Dickeya dadanantii, Dickeya solanii, Edwarsiellosis anguillarum, Edwarsiellosis ictaluri, Edwarsiellosis piscicida, Edwarsiellosis tarda, Enterobacterium catenabacteriul, Enterococcus faecalis, Erwinia amylovora, Escherichia coli, Francisella noatunensis, Francisella tularensis, Gallionella ferruginea, Klebsiella pneumoniae, Lactococcus garvieae, Legionella pneumophila, Mycobacterium fortuitum, Mycobacterium marinum, Nocardia asteroids, Nocardia crassostreae, Nocardia seriolae, Pantoea aglomerans, Pantoea ananatis, Pantoea stewartii, Pectobacterium atrosepticum, Pectobacterium carotovorum, Porphyromonas gingivalis, Proteus mirabilis, Pseudomonas aeruginosa, Pseudomonas anguilliseptica, Pseudomonas fluorescens, Pseudomonas savastanoi, Pseudomonas syringae, Renibacterium salmoninarum, Salmonella enterica, Serratia liquefaciens, Serratia marcescens, Shewanella japonica, Staphylococcus aureus, Staphylococcus epidermidis, Streptococcus agalactiae, Streptococcus iniae, Streptococcus mutans, Streptomyces scabiei, Thiobacillus ferooxidans, Vibrio cholerae, Vibrio harveyi, Vibrio parahaemolyticus, Vibrio vulnificus, Xanthomonas campestris, Xanthomonas citri, Xanthomonas oryzae, Xanthomonas translucens, Xylella fastidiosa, Yersinia pestis, Yersinia ruckeri.

In one particular embodiment, the bacteria may be resistant to treatment with one or more antimicrobial agents alone.

Thus, in the case where bacteria are resistant to treatment with one or more given antimicrobial agents, one or more other antimicrobial agents are administered in combination with the mutated lactonase as defined above and in particular with the mutated lactonase of sequence SEQ ID NO: 2. In this case, the use of the mutated lactonase as defined above and in particular with the mutated lactonase of sequence SEQ ID NO: 2 makes it possible to reduce the dose of antimicrobial agents necessary to eliminate these bacteria or inhibit their growth and thus to avoid the development of resistance mechanisms to these other antimicrobial agents by the bacteria.

In all aspects of the invention, said mutated lactonase of the invention is used at an effective dose.

In the present invention, “effective dose” means a sufficient dose of mutated lactonase to increase the susceptibility of bacteria to antimicrobial agents.

According to the present invention, the effective dose of mutated lactonase depends on the nature of the antimicrobial agents used and the bacteria to be killed.

According to the present invention, the mutated lactonase as defined above and in particular with the mutated lactonase of sequence SEQ ID NO: 2 can be used at a concentration of 0.1 mg/L to 10 g/L (liquid concentration) or 1 μg/cm² to 1 mg/cm² (solid surface concentration). In a particularly preferred embodiment, the mutated lactonase of the invention can be used at a concentration of 10 mg/L to 2 g/L.

In a particularly preferred embodiment, the mutated lactonase of the invention can be used at a concentration of 5 μg/cm² to 500 μg/cm².

In one embodiment, said anti-microbial agent is used at an effective dose.

In the present invention, “effective dose” means a dose of antimicrobial agents sufficient to kill said bacteria or inhibit their growth.

According to the present invention, the effective dose of antimicrobial agents depends on the bacteria to be eliminated.

In a particular embodiment, said anti-microbial agent may be used at a concentration of 10 μM to 100 mM.

In a particularly preferred embodiment, said anti-microbial agent may be used at a concentration of 1 mM to 100 mM.

In one embodiment, the effective dose of antimicrobial agents is decreased by at least a factor of 2 compared to the effective dose of said antimicrobial agents alone.

This means that, according to the present invention, the effective dose of antimicrobial agents is decreased by a factor of 2, 3, 4, 5, 10, 20, 30, 40, 50, 60, 70, 80, 90 or 100 compared to the effective dose of said antimicrobial agents alone.

In a particularly preferred embodiment, the effective dose of anti-microbial agents is decreased by a factor of from 2 to 20 compared to the effective dose of said anti-microbial agents alone.

In a second aspect, the invention relates to the use of a mutated lactonase, as defined in the first aspect, to inhibit bacterial growth.

Thus, in this second aspect, and in a particular embodiment, the invention relates to the use of a mutated lactonase belonging to the phosphotriesterase-like lactonase family in which at least the amino acid tryptophan located at the beginning of loop 8 is substituted by the amino acid isoleucine and of at least one antimicrobial agent to inhibit the growth of bacteria, wherein the inhibition of bacterial growth is increased by at least a factor of 2 over the use of said anti-microbial agent alone.

In this second aspect, and in another particular embodiment, the invention relates to the use of a mutated lactonase and at least one anti-microbial agent to inhibit the growth of bacteria, said mutated lactonase having the sequence SEQ ID NO: 1 in which amino acid W at position 263 is substituted by amino acid isoleucine I,

wherein the inhibition of bacterial growth is increased by at least a factor of 2 compared to the use of said at least one anti-microbial agent alone.

In the case of the present invention, the increase in number or mass of a given bacterium over a given period is referred to as “bacterial growth”. The measurement of bacterial growth can be measured according to methods known to the skilled person.

Bacterial inhibition” occurs when bacterial growth is stopped or reduced. Bacterial growth inhibition occurs when bacterial growth has been reduced by at least 10% compared to normal growth conditions and up to 100%.

In a particular embodiment, said mutated lactonase has the sequence SEQ ID NO: 2.

With respect to the anti-microbial agent, the various embodiments detailed in the first aspect apply to this second aspect.

Similarly, according to this second aspect, the bacteria are as detailed in the various embodiments of the first aspect of the invention.

As in the first aspect, said mutated lactonase and said at least one anti-microbial agent are used at an effective dose. In particular, the effective dose of antimicrobial agent may be decreased by at least a factor of 2 compared to the effective dose of said at least one antimicrobial agent alone.

In a third aspect, the invention relates to the use of a mutated lactonase, as defined in the first aspect, to increase the sensitivity of bacteria to bacteriophages.

Thus, in this third aspect, and in a particular embodiment, the invention relates to the use of a mutated lactonase belonging to the phosphotriesterase-like lactonase family in which at least the amino acid tryptophan located at the beginning of loop 8 is substituted by the amino acid isoleucine and at least one bacteriophage to increase the sensitivity of bacteria to bacteriophages, possibly naturally present in the environment or not, compared to the use of the said at least one bacteriophage alone.

In a particular embodiment, this third aspect relates to the use of a mutated lactonase and at least one bacteriophage to increase the sensitivity of bacteria to bacteriophages, possibly naturally present in the environment or not, compared to the use of said at least one bacteriophage alone, said mutated lactonase having the sequence SEQ ID NO: 1 in which amino acid W at position 263 is substituted by amino acid isoleucine I.

Thus, in a particular embodiment, said mutated lactonase has the sequence SEQ ID NO: 2.

As in the first aspect of the invention, in one embodiment, the sensitivity of the bacteria to the bacteriophage is increased by at least a factor of 2 compared to the use of the bacteriophage alone.

Thus, according to the present invention, when a given bacteriophage is used in combination with the mutated lactonase as defined above and in particular with the mutated lactonase of sequence SEQ ID NO: 2, the sensitivity of the bacteria to this bacteriophage is increased compared to the use of the same bacteriophage alone. This means that the amount of bacteriophage required to eliminate these bacteria or to inhibit their growth can be reduced when the bacteriophage is used with the mutated lactonase as defined above and in particular with the mutated lactonase of sequence SEQ ID NO: 2. This also avoids the development of resistance mechanisms to the bacteriophages by the bacteria.

In an embodiment of this third aspect, said bacteriophage may belong to the family Myoviridae, Siphoviridae, Podoviridae, Corticoviridae, Cystoviridae, Inoviridae, Leviviridae, Microviridae, Plasmaviridae and Tectiviridae or a cocktail thereof.

Similarly, according to this third aspect, the bacteria are as detailed in the various embodiments of the first aspect of the invention.

In one particular embodiment, the bacteria may be resistant to treatment with a bacteriophage alone.

As in the first aspect, said mutated lactonase and said bacteriophage are used at an effective dose. In particular, the effective dose of bacteriophage used may be reduced by at least a factor of 2 compared to the effective dose of bacteriophage alone.

In a fourth aspect, the invention relates to compositions comprising a mutated lactonase as defined in the first aspect.

Thus, in this fourth aspect, and in a particular embodiment, the invention concerns a composition comprising as active principle a mutated lactonase belonging to the phosphotriesterase-like lactonase family in which at least the amino acid tryptophan located at the beginning of loop 8 is substituted by the amino acid isoleucine and at least one antimicrobial agent,

the effective dose of antimicrobial agent in said composition being present in an amount at least 2 times lower than that of said at least one antimicrobial agent alone.

In this fourth aspect, and in a particular embodiment, the invention also relates to a composition comprising as active ingredient a mutated lactonase and at least one antimicrobial agent,

said mutated lactonase having the sequence SEQ ID NO: 1 in which amino acid W at position 263 is substituted by amino acid isoleucine I,

the effective dose of antimicrobial agent in said composition being present in an amount at least 2 times lower than that of said at least one antimicrobial agent alone.

Thus, in a particular embodiment, said mutated lactonase has the sequence SEQ ID NO: 2.

Thus, in a particular embodiment, the invention relates to a composition comprising as active principle a mutated lactonase belonging to the phosphotriesterase-like lactonase family in which at least the amino acid tryptophan located at the beginning of loop 8 is substituted by the amino acid isoleucine, and at least one antimicrobial agent,

and in particular said mutated lactonase having the sequence SEQ ID NO: 1 in which at least the amino acid W at position 263 is substituted by the amino acid isoleucine I, the effective dose of antimicrobial agent in said composition being present in an amount at least two times less than that of said at least one antimicrobial agent alone.

With respect to the anti-microbial agent, the various embodiments detailed in the first aspect apply to this fourth aspect.

Similarly, according to this fourth aspect, the bacteria are as detailed in the various embodiments of the first aspect of the invention.

As in the first aspect, said mutated lactonase and said anti-microbial agent are used at an effective dose.

In this fourth aspect and according to one embodiment, said anti-microbial agent is used at a concentration of 10 μM to 100 mM.

In a particularly preferred embodiment, said anti-microbial agent is used at a concentration of 1 mM to 100 mM.

Thus, the invention also relates to a composition as hereinbefore defined wherein said anti-microbial agent is used at a concentration of 10 μM to 100 mM, preferably 1 mM to 100 mM.

According to the present invention, the mutated lactonase as defined above and in particular with the mutated lactonase of sequence SEQ ID NO: 2 can be used at a concentration of 0.1 mg/L to 10 g/L (liquid concentration) or 1 μg/cm² to 1 mg/cm² (solid surface concentration).

In a particularly preferred embodiment, the mutated lactonase as defined above and in particular with the mutated lactonase of sequence SEQ ID NO: 2 can be used at a concentration of 10 mg/L to 2 g/L.

In a particularly preferred embodiment, the mutated lactonase as defined above and in particular with the mutated lactonase of sequence SEQ ID NO: 2 can be used at a concentration of 5 μg/cm² to 500 μg/cm².

Thus, the invention also relates to a composition as defined above wherein said mutated lactonase is used at a concentration of 0.1 mg/L to 10 g/L, preferably 10 mg/L to 2 g/L or at a concentration of 1 μg/cm² to 1 mg/cm², preferably 5 μg/cm² to 500 μg/cm².

In a particular embodiment, said composition described above may be applied to material contaminated or susceptible to contamination by said bacteria.

In a particular embodiment of this fourth aspect, said material contaminated with said bacteria or susceptible to being contaminated with said bacteria may be selected from:
medical devices such as dressings, catheters, endoscopes, implants, nebulizers
medical equipment
submerged surfaces such as ship hulls, harbour or oil infrastructures that can be the target of biofouling or biocorrosion,
industrial installations such as cooling towers, air-conditioning systems, bioreactors, piping, nebulizers, foggers, ponds.

In a particular embodiment, the invention also relates to a composition as described above for use in the prevention and/or treatment of pathologies related to bacterial infection.

In a particular embodiment, the invention also relates to a composition as described above as a plant protection product for the prevention and/or treatment of plant infections such as fire blight.

“Treatment” concerns the means of curing a declared disease, the symptoms of which are visible. “Prevention” concerns the means of preventing the occurrence of the condition.

In a particular embodiment, said composition described above can be used in animal health, in particular for the prevention and/or treatment of bacterial infections, the prevention and/or treatment of dysbiosis, the prevention and/or elimination of biofilms present in breeding ponds and aquariums.

In a particular embodiment, the invention also relates to a composition as described above as a food supplement for humans or animals or as an animal nutrition product.

In a particular embodiment, said composition described above can be used in human health, in particular for the prevention and/or treatment of bacterial infections such as pneumonia, nosocomial diseases, wounds, burns, eye infections, diabetic foot, for the prevention and/or treatment of dysbiosis, or for the prevention and/or treatment of dental plaque.

In a particular embodiment, the invention relates to a composition as defined above comprising as active ingredient a mutated lactonase belonging to the phosphotriesterase-like lactonase family in which at least the amino acid tryptophan located at the beginning of loop 8 is substituted by the amino acid isoleucine and at least one antimicrobial agent,

in particular, said mutated lactonase having the sequence SEQ ID NO: 1 in which the amino acid W at position 263 is substituted by the amino acid isoleucine I,

the effective dose of antimicrobial agent in said composition being present in an amount at least two times less than that of said at least one antimicrobial agent alone, for its use:
in animal health, in particular for the prevention and/or treatment of bacterial infections, the treatment of dysbiosis, the prevention of biofilms present in breeding ponds and aquariums, or
in human health, in particular for the prevention and/or treatment of bacterial infections such as pneumonia, nosocomial diseases, wounds, burns, eye infections, diabetic foot, for the prevention and/or treatment of dysbiosis, or for the prevention and/or treatment of dental plaque.

In a particular embodiment, said composition described above may be formulated with at least one suitable excipient for use as a solution, oil, suspension, emulsion, nanoparticles, liposomes, granules or functionalized surface.

In a fifth aspect, the invention relates to a method of preventing and/or treating pathologies related to bacterial infections, comprising the administration of a mutated lactonase as defined in the first aspect and at least one antimicrobial agent.

Thus, in this fifth aspect and in a particular embodiment, the invention relates to a method of preventing and/or treating pathologies related to bacterial infections, comprising the administration of a mutated lactonase belonging to the phosphotriesterase-like lactonase family in which at least the amino acid tryptophan located at the beginning of loop 8 is substituted by the amino acid isoleucine and of at least one antimicrobial agent,
the effective dose of antimicrobial agents in said composition being present in an amount at least 2 times lower than that of said at least one antimicrobial agent alone.

In this fifth aspect and in a particular embodiment, the invention relates to a method of preventing and/or treating pathologies related to bacterial infections, comprising the administration of a mutated lactonase and at least one antimicrobial agent,

said mutated lactonase having the sequence SEQ ID NO: 1 in which amino acid W at position 263 is substituted by amino acid isoleucine I,

the effective dose of antimicrobial agents in said composition being present in an amount at least 2 times lower than that of said at least one antimicrobial agent alone.

In an embodiment of this fifth aspect, said bacterial infections may be bacterial infections in plants such as fire blight.

In an embodiment of this fifth aspect, said bacterial infections may be bacterial infections in animals such as dysbiosis.

In an embodiment of this fifth aspect, said bacterial infections may be bacterial infections in humans such as pneumonia, nosocomial diseases, wounds, burns, eye infections, diabetic foot, for the treatment of dysbiosis, or for the treatment of dental plaque.

All the embodiments of the various aspects described above are applicable to this fifth aspect.

The following figures and examples illustrate the invention, without limiting its scope.

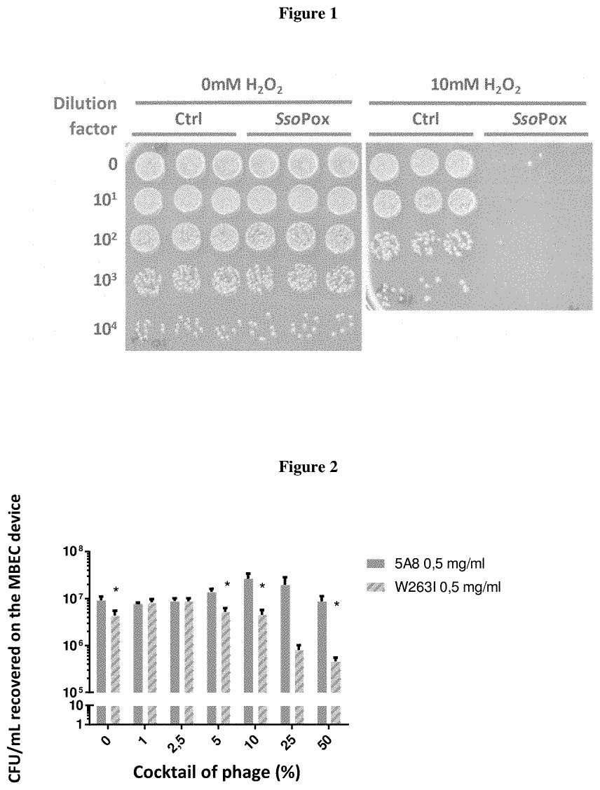

FIG. 1: Enumeration of P. aeruginosa bacteria recovered from biofilms formed in the presence or absence of SsoPox-W263I lactonase after treatment with the antiseptic H2O2.

Left: Enumeration of P. aeruginosa bacteria recovered from biofilms in the absence of H2O2 antiseptic treatment, in the absence of SsoPox-W263I lactonase (Ctrl) or in the presence of SsoPox-W263I lactonase (SsoPox)

Right: Enumeration of P. aeruginosa bacteria recovered from biofilms after treatment with 10mM H2O2 antiseptic, in the absence of SsoPox-W263I lactonase (Ctrl) or in the presence of SsoPox-W263I lactonase (SsoPox)

FIG. 2: Increased susceptibility of Pseudomonas aeruginosa PA14 to Intesti phage cocktail. Bars represent the number of bacteria after exposure to different concentrations of the phage cocktail (ranging from 0 to 50% (vol/vol)), treated with the mutated lactonase W263I (0.5mg/mL) or with the inactive enzyme SsoPox-5A8 (0.5mg/mL).

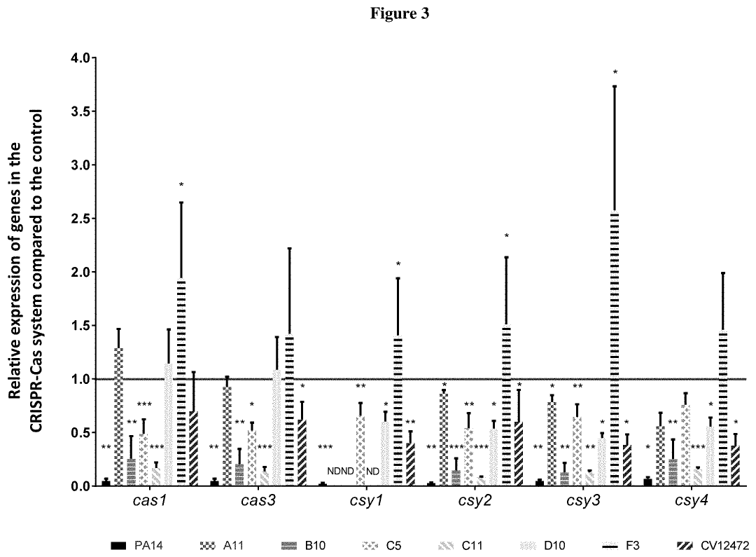

FIG. 3: Expression changes of the CRISPR-Cas system. Expression of the CRISPR-Cas system genes casl, cas3, csy1, csy2, csy3 and csy4 was measured for the P. aeruginosa model strain PA14 as well as for clinical isolates from diabetic foot infections (All, B10, C5, C11, D10 and F3) and for the marine bacterium Chromobacterium violaceum CV12472. Cultures were treated with the enzyme SsoPox-W263I or the inactive enzyme SsoPox-5A8 (V27 G/P67 Q/L72C/Y97 S/Y99A/T 177D/R223 L/L226Q/L228 M/W263H). Histograms represent the expression of genes in the CRISPR-Cas system treated with SsoPox-W263I normalised to the values obtained with the inactive variant. Error bars represent variations for 2 technical duplicates of 2 biological replicates. *p-values<0.05, *p-values<0.01, ***p-values<0.001 by Student's t-test. ND indicates that the expression was not detected.

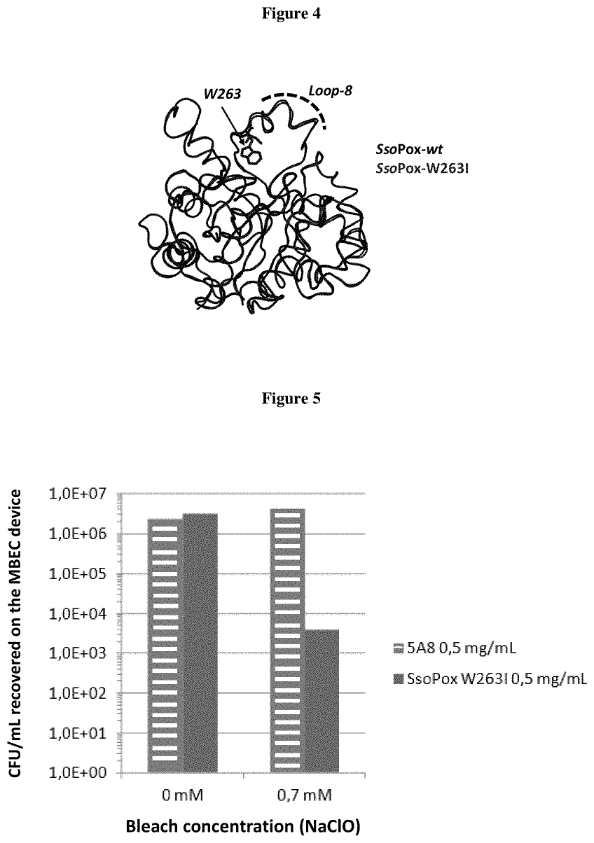

FIG. 4: Three-dimensional structure of phosphotriesterase-like lactonase enzymes. The three-dimensional structure of the phosphotriesterase-like lactonase enzymes is conserved. The mutated tryptophan residue corresponds to the tryptophan located at the beginning of loop 8 of the Phosphotriesterase-Like Lactonase family enzymes. In the case of the present invention, the mutated lactonase of SEQ ID NO: 2 (SsoPox W263I) mutated tryptophan residue is located at the beginning of loop 8 corresponds to the tryptophan at position 263 of the primary structure.

FIG. 5: Enumeration of P. aeruginosa bacteria recovered from biofilms formed in the presence or absence of SsoPox-W263I lactonase after treatment with NaOCl bleach.

Left: Enumeration of P. aeruginosa bacteria recovered from biofilms in the absence of NaOCl bleach treatment, in the presence of SsoPox-W263I lactonase (solid bar) or an inactive variant of the SsoPox 5A8 enzyme (hatched bar).

Right: Enumeration of P. aeruginosa bacteria recovered from biofilms after treatment with 0.7mM NaOCl bleach in the presence of SsoPox-W263I lactonase (solid bar) or an inactive variant of the SsoPox 5A8 enzyme (hatched bar).

A 2 log decrease is observed when bleach (0.7 mM) and SsoPox-W263I lactonase are used simultaneously demonstrating the synergistic effect of the combination.

Material and Method

a) Sensitivity Tests

The doses of antimicrobial agents required to eliminate bacterial biofilms are determined using the “MBEC (Minimal Biofilm Eradication Concentration) Assay TM” technique developed by Innovotech (Alberta, Canada) according to the supplier's data.

Bacterial biofilms are formed by bacterial growth in a medium and conditions adapted to the bacteria studied in the presence or absence of the lactonase SsoPox-W263I of sequence SEQ ID NO: 2.

Bacteria are pre-cultured for 6 hours in oxygenated flasks under the conditions indicated in Table 1 and then MBEC plates are plated by diluting the pre-culture to 1:1000 in the presence or absence of SsoPox-W263I lactonase at 0.5 mg/mL. After 24 hours of growth, the bacterial biofilms formed on the spikes of the MBEC plate cover are rinsed by immersion for 5 minutes in a buffer solution (Table 1). The biofilms are then immersed in a buffer solution containing antimicrobial agents (disinfectants, bactericidal or bacteriostatic antibiotics, bacteriophages, biocides) for a period of time representative of the mechanism of action of the antimicrobial agent studied (1.5 h for antiseptics, 3 h for antibiotics, 4 h for phages). After immersion in the antimicrobial agents, the bacterial biofilms were rinsed for 5 minutes in a buffer solution and then incubated for 1 hour in a nutrient medium adapted to the bacteria studied and containing detergents to detach the biofilms (Table 1). After 1 hour of incubation, the bacteria detached from the biofilm and thus present in the wells of the MBEC plate are serially diluted and plated on suitable nutrient agar to perform bacterial counts and determine the number of bacteria that survived the combined treatment of the mutated SsoPox-W263I enzyme and the antimicrobial agent (FIG. 1). The MBEC is the minimum concentration of antimicrobial agent to eradicate the bacteria in the biofilm.

TABLE 2
Experimental conditions for the determination of MBEC.

| | | Pre- | Nutrient | | | | |
|---|---|---|---|---|---|---|---|
| | | culture | medium | | | Buffer | Recovery |
| Bacteria | Strain | conditions | MBEC | Temperature | Agitation | solution | environment |
| Pseudomonas | UCBPP- | LB - | MOPS | 37° C. | Orbital - | MOPS | Recovery |
| aeruginosa | PA14 | 37° C. | glutamate | | 110 RPM | | LB |
| Chromobacterium | ATCC- | LB - | LB | 25° C. | Orbital - | PBS | Recovery |
| violaceum | 12472 | 37° C. | | | 110 RPM | | LB |

LB (10 g/L peptone, 5 g/L yeast extract, 10 g/L NaCl); 10x MOPS buffer (500 mM MOPS, 40 mM Tricine, 500 mM NaCl, 10 mM K2HSO4, 500 mM MgCl2, 100 mM CaCl2, 3 mM (NH4)6Mo7O24, 400 mM H3BO3, 30 mM Co(OAc)2, 10 mM CuSO4, 80 mM MnSO4, 10 mM ZnSO4 [pH 7.0] , sterilised by 0.22 μm filtration); MOPS glutamate medium (MOPS 1x, 15 mM NH4Cl, 5 μM Fe2SO4, 4 mM K2HPO4, 25 mM glutamate); PBS (8 g/L NaCl, 0.2 g/L KCl, 1.44 g/L Na2HPO4, 0.24 g/L KH2PO4); Recovery LB (LB, 20 g/L saponin, 10 g/L Tween-80)

b) Gene Expression of the CRISPR-Cas System

Bacteria were grown in MOPS medium for P. aeruginosa and LB for C. violaceum, in the presence of the mutated lactonase SsoPox-W263I (0.5 mg/mi) or its inactive variant 5A8 (0.5 mg/mi). After 16 hours of culture (stationary phase), the bacteria were recovered by centrifugation.

RNAs were extracted and purified with the PureLink® RNA mini kit (ThermoFisher) according to the supplier's recommendations and then treated with the TURBO DNA-free™ kit (ThermoFisher) to remove genomic DNA contamination. Samples were checked for quality by 1.5% agarose gel migration and the amount of nucleic acid was measured with a NanoDrop 2000 spectrophotometer (Thermo Scientific) at OD260 nm. Complementary DNAs (cDNAs) were synthesised using the TaqMan® Reverse Transcription Reagents kit (ThermoFisher) according to the manufacturer's recommendations. RT-PCR was then performed using the LuminoCt® SYBR® Green qPCR ReadyMix™ kit and a CFX thermocycler (Bio-Rad) and specific primer pairs. PCR amplification was performed with the following method: Denaturation for 5 minutes at 94° C., followed by 29 cycles of [1 minute at 94° C., 1 minute at 55° C., 30 seconds at 72° C.] for amplification, then a final elongation step for 7 minutes at 72° C. Sample fluorescence is measured at the end of each cycle and denaturation curves were analysed with CFX Manager™ software (Bio-Rad). Gene expression was normalised by expression of a 5S RNA housekeeping gene.

TABLE 3
Sequences of primers used to assess gene
expression in the CRISPR-Cas system

| Bacterial | Target | |
|---|---|---|
| species | gene | Sequence |
| P. | cas1 | Forward TCAAGGACTCGCTGATCCTG |
| aeruginosa | | (SEQ ID NO: 3) |
| | | Reverse GATCATGAAGTCCAGGGCCT |
| | | (SEQ ID NO: 4) |
| | cas3 | Forward GGTTGATCGTCAGCCATCAT |
| | | (SEQ ID NO: 5) |
| | | Reverse GGCCTTTTCTTTTGCGTCT |
| | | (SEQ ID NO: 6) |
| | csy1 | Forward TCTTCGAGCATGACTTCGGA |
| | | (SEQ ID NO: 7) |
| | | Reverse TGGCGAGGTTGTTATGGACT |
| | | (SEQ ID NO: 8) |
| | csy2 | Forward CGTCCGAAGAAGAAGCATCG |
| | | (SEQ ID NO: 9) |
| | | Reverse CGCAGCGGTGTTTCTCTATC |
| | | (SEQ ID NO: 10) |
| | csy3 | Forward AAGACCAAGGACCGTGACC |
| | | (SEQ ID NO: 11) |
| | | Reverse AGCCCTGATCGTTCACGTAG |
| | | (SEQ ID NO: 12) |
| | csy4 | Forward ACAGGATCGGCGTGAGCTT |
| | | (SEQ ID NO: 13) |
| | | Reverse CCGCAACCCTTCCAGCCA |
| | | (SEQ ID NO: 14) |
| | 5S | Forward GAACCACCTGATCCCTTCCC |
| | | (SEQ ID NO: 15) |
| | | Reverse TAGGAGCTTGACGATGACCT |
| | | (SEQ ID NO: 16) |
| C. | cas1 | Forward CAGGATGGCTGCGTCTTTG |
| violaceum | | (SEQ ID NO: 17) |
| | | Reverse AACTACCTGGCCTACGGC |
| | | (SEQ ID NO: 18) |
| | cas3 | Forward GGACAGGTAGGAGGCTTG |
| | | (SEQ ID NO: 19) |
| | | Reverse TACGCGAGCAAGTGACCC |
| | | (SEQ ID NO: 20) |
| | csy1 | Forward GGAATTCCGCCTCCGCCA |
| | | (SEQ ID NO: 21) |
| | | Reverse GCCGACAGCGATGAAGAC |
| | | (SEQ ID NO: 22) |
| | csy2 | Forward TCACCGGCCTGATGACGGC |
| | | (SEQ ID NO: 23) |
| | | Reverse GAAGCGCTGGATGTAGTCG |
| | | (SEQ ID NO: 24) |
| | csy3 | Forward GCGAGTACAGGCTTTCCAC |
| | | (SEQ ID NO: 25) |
| | | Reverse AAACCATCTGGCGGCACTC |
| | | (SEQ ID NO: 26) |
| | csy4 | Forward CGGAAGCATTGGCCGGTG |
| | | (SEQ ID NO: 27) |
| | | Reverse CGCGCGACAGGCTGATG |
| | | (SEQ ID NO: 28) |
| | 5S | Forward CTGGTGGCCATAGCGAGG |
| | | (SEQ ID NO: 29) |
| | | Reverse GTCTGGCGGTGTCCTACTT |
| | | (SEQ ID NO: 30) |

Results

a) Sensitivity Tests

FIG. 1 shows that without antiseptic treatment (left), the same number of bacteria are recovered from biofilms whether or not there has been treatment with SsoPox-W263I mutated lactonase. After treatment for 1.5 hours with 10 mM H2O2 antiseptic, no bacteria are recovered from the biofilm with the use of the SsoPox-W263I mutated lactonase whereas 104-105 bacterial cells are recovered without the lactonase. In the control sample (ctrl) made with an inactive variant of SsoPox-5A8 (V27G/P67Q/L72C/Y97S/Y99A/T177D/R223L/L226Q/L228M/W263H), 100 mM of antiseptic is required to completely eradicate the biofilm.

This means that the use of antiseptic and SsoPox-W263I mutated lactonase significantly reduces the number of bacteria recovered from the biofilm compared to the use of antiseptic alone or SsoPox-W263I mutated lactonase alone.

The results obtained in Table 3 show that the presence of the mutated lactonase SsoPox-W263I decreases by a factor of 10 the concentration of antibiotic and antiseptic (gentamicin, tobramycin and H2O2) required to eliminate the biofilms of P. aeruginosa. The same trend, with a factor of at least 20, is observed for a biofilm of the marine bacterium C. violaceum treated with a biocide used in antifouling paints for ships' hulls.

The preventive use of the mutated lactonase SsoPox-W263I, in addition to the biocide, therefore significantly reduces the use of biocidal products that have a negative impact on the environment and are known to facilitate the emergence of resistant bacteria in hospital or natural environments.

TABLE 4
MBEC with or without the use of the SsoPox W263I mutated lactonase

| | | Type of | | SsoPox | | |
|---|---|---|---|---|---|---|
| Bacteria | Strain | biocide | Name of the biocide | W263I | MBEC | |
| Pseudomonas | UCBPP- | Antibiotic | Gentamicin | — | 20 | μg/mL |
| aeruginosa | PA14 | | | 0.5 mg/mL | 2 | μg/mL |
| Pseudomonas | UCBPP- | Antibiotic | Tobramycin | — | 10 | μg/mL |
| aeruginosa | PA14 | | | 0.5 mg/mL | 1 | μg/mL |
| Pseudomonas | UCBPP- | Antiseptic | H2O2 | — | 100 | mM |
| aeruginosa | PA14 | | | 0.5 mg/mL | 10 | mM |
| Chromobacterium | ATCC- | Broad | Preventol A4S | — | <200 | μM |
| violaceum | 12472 | spectrum | (Dichlofluanide) | 0.5 mg/mL | >10 | μM |
| | | biocide | | | | |

In addition, Pseudomonas aeruginosa bacteria are also treated with W263I mutated lactonase and a bacteriophage cocktail (Instesti cocktail; Microgen Russia) with satisfactory results.

P. aeruginosa PA14 is treated with the mutated enzyme SsoPox-W263I and the Intesti phage cocktail or with the Intesti phage cocktail and the inactive variant SsoPox-5A8. The Intesti phage cocktail consists of a mixture of sterile filtrates of phages directed against Shigella flexneri (serovariants 1, 2, 3, 4, 6), Shigellasi, Proteus vulgaris, Proteus mirabilis, Enterococcus, Staphylococcus, Pseudomonas aeruginosa and excipients such as 8-hydroxyquinoline sulfate monohydrate at 0.0001 g/ml (estimated content) and is marketed by Intesti-bacteriophage, Microgen, Russia

FIG. 2 shows that P. aeruginosa PA14 treated with the mutated enzyme SsoPox-W263I and the Intesti phage cocktail is more sensitive to the Intesti phage cocktail than the bacteria treated with the inactive variant SsoPox-5A8 and the Intesti phage cocktail. Indeed, the bacteria treated with the inactive SsoPox-5A8 enzyme and the phage cocktail is little impacted by the Intesti phage cocktail whereas less bacteria are counted after treatment with the mutated SsoPox-W263I enzyme.

b) Gene Expression of the CRISPR-Cas System

The CRISPR-Cas system is involved in the defence of bacteria against bacteriophages. To determine whether the SsoPox-W263I mutated enzyme affects the regulation of the CRISPR-Cas system, the expression levels of the CRISPR-Cas genes cas 1, cas3, csy 1, csy2, csy3 and csy4 were measured in P. aeruginosa PA14 and clinical isolates from diabetic foot infections (All, B11, B12 and B13). aeruginosa PA14 and clinical isolates of P. aeruginosa from diabetic foot infections (All, B10, C5, C11, D10, F3) as well as in the marine strain of Chromobacterium violaceum CV12472. Primers targeting these different genes were created from the genomes of P. aeruginosa PA14 and C. violaceum CV12472 (Table 2). Cultures were treated with the enzyme SsoPox-W263 or the inactive enzyme SsoPox-5A8 (V27 G/P67 Q/L72C/Y97 S/Y99A/T177D/R223L/L226Q/L228M/W263 H). Gene expression is completely abolished in P. aeruginosa PA14 after treatment with the SsoPox-W263I mutated enzyme. In B10 and Cll gene expression is decreased by a factor of 5.5 and 8 respectively. In All and D10 the expression of the csyl-4 genes was significantly reduced. In F3, on the other hand, the expression of the genes is increased by a factor of 1.7 on average. In C. violaceum the expression of the cas3 and csy2-4 genes was significantly reduced. These results show that the SsoPox-W263I enzyme impacts the regulation of the CRISPR-Cas system.

c) Demonstration of a synergistic effect between the W263I mutated lactonase and a biocide (NaOCl)

It is shown in FIG. 5, that without treatment with sodium hypochlorite (NaOCl) (left), the same number of bacteria are recovered from biofilms whether or not there was treatment with W263I mutated lactonase. After treatment with 0.7 mM sodium hypochlorite and W263I mutated lactonase, the number of bacteria recovered from biofilms was reduced by 2 Log compared to the use of sodium hypochlorite alone.

These results show that the use of sodium hypochlorite in combination with the W263I mutated lactonase significantly reduces the number of bacteria recovered from the biofilm compared to the use of sodium hypochlorite alone or W263I mutated lactonase alone, thus demonstrating a synergistic effect between sodium hypochlorite and W263I mutated lactonase.

1. A method for increasing susceptibility of bacteria to antimicrobial agents, comprising exposing the bacteria to a mutated lactonase belonging to the phosphotriesterase-like lactonase family,
wherein at least the amino acid tryptophan at the beginning of loop 8 is substituted by the amino acid isoleucine.

2. The method of claim 1, wherein the antimicrobial agent is selected from the group consisting of antibiotics or a mixture of antibiotics, disinfectants or a mixture of disinfectants, biocides or a mixture of biocides and bacteriophages possibly naturally present in the environment or not, or a cocktail of such bacteriophages.

3. The method of claim 2, wherein said antibiotic is selected from the group consisting of: Amikacin, Amoxicillin, Amoxicillin/clavulanate, Ampicillin, Amprolium, Apramycin, Aspoxicillin, Aureomycin, Avilamycin, Azithromycin, Bacitracin, Bambermycin, Baquiloprim, Benzylpenicillin, Bicozamycin, Carbadox, Cefacetrile, Cefalexin, Cefalonium, Cefalotin, Cefapyrin, Cefazolin, Cefdinir, Cefquinome, Ceftiofur, Ceftriaxone, Cefuroxime, Chloramphenicol, Chlortetracycline, Ciprofloxacin, Clarithromycin, Clindamycin, Cloxacillin, Colistin, Dalbavancin, Danofloxacin, Decoquinate, Diclazuril, Dicloxacillin, Difloxacin, Doripenem, Doxycycline, Enramycin, Enrofloxacin, Ertapenem, Erythromycin, Florfenicol, Flumequine, Fosfomycin, Framycetin, Fusidic acid, Gentamicin, Gentamicin Sulfate, Gramicidin, Halofuginone hydrobromide, Hetacillin, Imipenem, Imipenem/cilastatin, Josamycin, Kanamycin, Kitasamycin, Laidlomycin, Lasalocid , Levofloxacin, Lincomycin, Lincomycin hydrochloride, Maduramycin, Marbofloxacin, Mecillinam, Meropeneme, Miloxacin, Minocycline, Mirosamycin, Monensin, Moxifloxacin, Nafcillin, Nalidixic acid, Narasin, Neomycin, Neomycin/oxytetracycline, Neosporin, Nicarbazine, Norfloxacin, Novobiocin, Ofloxacin, Orbifloxacin, Oritavancin, Oxacillin, oxolinic acid, Oxytetracycline, Paromomycin, penethamate hydroxide, Penicillin, Penicillin G Potassium, Penicillin procaine, Penicillin V potassium, Phenethicillin, Phenoxymethylpenicillin, Pirlimycin, Polymyxin, Polymyxin B, Polysporin (bacitracin/polymyxin), Pristinamycin, Rifampin, Rifaximin, Roxarsone, Salinomycin, Semduramicin, Spectinomycin, Spiramycin, Streptomycin, Sulfachlorpyridazine, Sulfadiazine, Sulfadimerazine, Sulfadimethoxazole, Sulfadimethoxine, Sulfadimethoxine and ormetoprim 5:3, Sulfadimidine, Sulfadoxine, Sulfafurazole, Sulfaguanidine, Sulfamethazine, Sulfamethoxazole/trimethoprim, Sulfamethoxine, Sulfamethoxypyridazine, Sulfamonomethoxine, Sulfanilamide, Sulfaquinoxaline, Sulfasalazine, Sulfisoxazole, Surfactin, Telavancin, Terdec amyc in, Tetracycline, Thiamphenicol, Tiamulin, Ticarcillin, Tilmicosin, Tobicillin, Tobramycin, Trimethoprim, Trimethoprim/Sulfonamide, Tulathromycin, Tylo sin, Valnemulin, Vancomycin, Virginiamycin.

4. The method of claim 2, wherein said disinfecting agent comprises an alcohol, chlorine, aldehyde, oxidising agent, iodine, ozone, phenolic compound, quaternary ammonium compound or a mixture of two or more of these.

5. The method of claim 2, wherein the biocide is selected from the group consisting of: biocidally active peroxides, mono- and polyhydric alcohols, aldehydes, acids, ozone, naphtha compounds and compounds containing an alkali metal, transition metal, group III or group IV metal, sulphur, nitrogen or halogen atom and mixtures of two or more of these.

6. The method of claim 2, wherein the bacteriophage belongs to the family Myoviridae, Siphoviridae, Podoviridae, Corticoviridae, Cystoviridae, Inoviridae, Leviviridae, Microviridae, Plasmaviridae and Tectiviridae or a cocktail thereof.

7. The method of claim 1, wherein the effective dose of antimicrobial agent is decreased by at least a factor of 2 compared to the effective dose of antimicrobial agent alone.

8. Composition comprising as active principle a mutated lactonase belonging to the phosphotriesterase-like lactonase family in which at least the amino acid tryptophan located at the beginning of loop 8 is substituted by the amino acid isoleucine, and at least one antimicrobial agent.

9. The composition of claim 8, wherein said anti-microbial agent is used at a concentration of 10 μM to 100 mM.

10. The composition according to claim 8, wherein said mutated lactonase is used at a concentration of 0.1 mg/L to 10 g/L.

11. A method for removing bacteria from a material contaminated or susceptible to contamination by said bacteria, comprising applying to the material the composition of claim 8,
wherein said material contaminated with said bacteria or liable to be so is selected from:
medical devices:
medical equipment:
submerged surfaces; and
industrial installations.

12. A plant protection product suitable for the prevention and/or treatment of plant infections, the plant protection product comprising the composition of claim 8.

13. A food supplement for humans or animals or an animal nutrition product comprising the composition of claim 8.

14. A solution, oil, suspension, emulsion, nanoparticle, liposome, granule or functionalized surface comprising the composition of claim 8 formulated with at least suitable excipient.

15. A method for
in animal health, the prevention and/or treatment of bacterial infections, the treatment of dysbiosis, the prevention of biofilms present in breeding tanks and aquariums, or
in human health, the prevention and/or treatment of bacterial infections, nosocomial diseases, wounds, burns, eye infections, diabetic foot, for the prevention and/or treatment of dysbiosis, or for the prevention and/or treatment of dental plaque
the method comprising administering a therapeutically effective dose, to a patient in need thereof, of a composition comprising as active principle a mutated lactonase belonging to the phosphotriesterase-like lactonase family in which at least the amino acid tryptophan located at the beginning of loop 8 is substituted by the amino acid isoleucine and at least one antimicrobial agent.

16. The method of claim 1, wherein said mutated lactonase has the sequence SEQ ID NO: 1 in which at least the amino acid tryptophan W at position 263 is substituted by the amino acid isoleucine I.

17. The method of claim 16, wherein said sensitivity of bacteria to antimicrobial agents is increased by at least a factor of 2.

18. The composition of claim 8, wherein said mutated lactonase has the sequence SEQ ID NO: 1 in which at least the amino acid W at position 263 is substituted by the amino acid isoleucine I.

19. The composition according to claim 8, wherein said mutated lactonase is used at a concentration of 1 μg/cm² to 1 mg/cm².

20. A method for removing bacteria from a material contaminated or susceptible to contamination by said bacteria, comprising applying to the material the composition of claim 9,
wherein said material contaminated with said bacteria or liable to be so is selected from:
medical devices;
medical equipment;
submerged surfaces; and
industrial installations.


SEQUENCE LISTING (30 sequences)

SEQ ID NO: 1 - protein, 314 aa, Sulfolobus solfataricus
MRIPLVGKDSIESKDIGFTLIHEHLRVFSEAVRQQWPHLYNEDEEFRNAVNEVKRAMQFGVKTIVDPTVMGLGRDIRFMEKVVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPKLAPRWSITLIFEDTIPFLKRNGVNEEVIATIFKENPKKFFS

SEQ ID NO: 2 - protein, 314 aa, Sulfolobus solfataricus
MRIPLVGKDSIESKDIGFTLIHEHLRVFSEAVRQQWPHLYNEDEEFRNAVNEVKRAMQFGVKTIVDPTVMGLGRDIRFMEKVVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDIGTAKPEYKPKLAPRWSITLIFEDTIPFLKRNGVNEEVIATIFKENPKKFFS

SEQ ID NO: 3 - DNA, 20 bp, Artificial sequence
  amorce
tcaaggactcgctgatcctg

SEQ ID NO: 4 - DNA, 20 bp, Artificial sequence
  amorce
gatcatgaagtccagggcct

SEQ ID NO: 5 - DNA, 20 bp, Artificial sequence
  amorce
ggttgatcgtcagccatcat

SEQ ID NO: 6 - DNA, 19 bp, Artificial sequence
  amorce
ggccttttcttttgcgtct

SEQ ID NO: 7 - DNA, 20 bp, Artificial sequence
  amorce
tcttcgagcatgacttcgga

SEQ ID NO: 8 - DNA, 20 bp, Artificial sequence
  amorce
tggcgaggttgttatggact

SEQ ID NO: 9 - DNA, 20 bp, Artificial sequence
  amorce
cgtccgaagaagaagcatcg

SEQ ID NO: 10 - DNA, 20 bp, Artificial sequence
  amorce
cgcagcggtgtttctctatc

SEQ ID NO: 11 - DNA, 19 bp, Artificial sequence
  amorce
aagaccaaggaccgtgacc

SEQ ID NO: 12 - DNA, 20 bp, Artificial sequence
  amorce
agccctgatcgttcacgtag

SEQ ID NO: 13 - DNA, 19 bp, Artificial sequence
  amorce
acaggatcggcgtgagctt

SEQ ID NO: 14 - DNA, 18 bp, Artificial sequence
  amorce
ccgcaacccttccagcca

SEQ ID NO: 15 - DNA, 20 bp, Artificial sequence
  amorce
gaaccacctgatcccttccc

SEQ ID NO: 16 - DNA, 20 bp, Artificial sequence
  amorce
taggagcttgacgatgacct

SEQ ID NO: 17 - DNA, 19 bp, Artificial sequence
  amorce
caggatggctgcgtctttg

SEQ ID NO: 18 - DNA, 18 bp, Artificial sequence
  amorce
aactacctggcctacggc

SEQ ID NO: 19 - DNA, 18 bp, Artificial sequence
  amorce
ggacaggtaggaggcttg

SEQ ID NO: 20 - DNA, 18 bp, Artificial sequence
  amorce
tacgcgagcaagtgaccc

SEQ ID NO: 21 - DNA, 18 bp, Artificial sequence
  amorce
ggaattccgcctccgcca

SEQ ID NO: 22 - DNA, 18 bp, Artificial sequence
  amorce
gccgacagcgatgaagac

SEQ ID NO: 23 - DNA, 19 bp, Artificial sequence
  amorce
tcaccggcctgatgacggc

SEQ ID NO: 24 - DNA, 19 bp, Artificial sequence
  amorce
gaagcgctggatgtagtcg

SEQ ID NO: 25 - DNA, 19 bp, Artificial sequence
  amorce
gcgagtacaggctttccac

SEQ ID NO: 26 - DNA, 19 bp, Artificial sequence
  amorce
aaaccatctggcggcactc

SEQ ID NO: 27 - DNA, 18 bp, Artificial sequence
  amorce
cggaagcattggccggtg

SEQ ID NO: 28 - DNA, 17 bp, Artificial sequence
  amorce
cgcgcgacaggctgatg

SEQ ID NO: 29 - DNA, 18 bp, Artificial sequence
  amorce
ctggtggccatagcgagg

SEQ ID NO: 30 - DNA, 19 bp, Artificial sequence
  amorce
gtctggcggtgtcctactt